(19) United States
(12) Patent Application Publication
(10) Pub. No.: US 2009/0098212 A1
(43) Pub. Date: Apr. 16, 2009

(54) Acoustically sensitive drug delivery particle

(75) Inventors: Sigrid Fossheim, Oslo (NO); Cecilia Leal Lauten, Oslo (NO); Petras Juzenas, Gjettum (NO); Karen Sibylla Rognvaldsson, Oslo (NO); Esben A. Nilssen, Oslo (NO)

Correspondence Address: BIRCH STEWART KOLASCH & BIRCH, PO BOX 747, FALLS CHURCH, VA, 22040-0747, US

(73) Assignee: EPITARGET AS, Oslo (NO)

(21) Appl. No.: 12/285,120

(22) Filed: Sep. 29, 2008

Related U.S. Application Data
(63) Continuation-in-part of application No. PCT/NO2008/000115, filed on Mar. 28, 2008.

(30) Foreign Application Priority Data
Mar. 30, 2007 (NO) 20071688
Jun. 7, 2007 (NO) 20072822

(51) Int. Cl. A61K 9/50; A61K 47/00

(52) U.S. Cl. 424/502; 514/785

(57) ABSTRACT

Disclosed are ultrasound sensitive drug carrying particles, uses and methods thereof, wherein the drug carrying particles accumulate in the diseased target tissue and efficiently release their payload upon ultrasound exposure.

### FIELD OF THE INVENTION

The present invention relates to an acoustically sensitive drug delivery particle allowing efficient release of a drug in a defined volume or area in a mammal. More particularly, the invention relates to acoustically sensitive drug carrying particles, e.g. liposomes, as well as compositions, methods and uses thereof.

### BACKGROUND OF THE INVENTION

A serious limitation of traditional medical treatment is lack of specificity, that is, drugs do not target the diseased area specifically, but affect essentially all tissues. This limitation is particularly evident in chemotherapy where all dividing cells are affected imposing limitations on therapy. One strategy to achieve improved drug delivery is incorporation or encapsulation of drugs in e.g. liposomes, plurogels and polymer particles. The rationale behind this strategy has been to improve the therapeutic-to-toxicity ratio by protecting the patient from potential toxic side effects, as well as taking advantage of the so-called enhanced permeability and retention effect (EPR) (Maeda H, Matsumura Y., Crit. Rev. Ther. Drug Carrrier Syst., 6:193-210, 1989) to obtain passive accumulation of drugs in target tissue. Several liposomal cytotoxic drugs are already commercially available like e.g. liposomal doxorubicin (CAELYX® and DOXIL®). However, there are still disadvantages associated with such liposome products and the therapeutic-to-toxicity ratio is borderline. One challenge is to engineer particles with both optimal release characteristics and reduced toxicity: efficient shielding of the (toxic) drug in blood circulation usually implies suboptimal release rates in the target tissue, and vice versa. Ultrasound (US) mediated drug release has been proposed as one solution to this problem (for a review, see Pitt et al, Expert Opin Drug Deliv, 2004; 1 (1): 37-56). Here US sensitive drug carriers are allowed to accumulate in the target tissue before the payload is released by means of therapeutic ultrasound. The fact that ultrasound also facilitates intracellular uptake of the drug further improves the therapeutic effect (Larina I V, Evers B M, et al. Technol. Cancer Res. Treat, 4:217-226, 2005). Four main types of ultrasound responsive carriers have been described so far 1) micelles, 2) gas-containing liposomes, 3) microbubbles, and 4) liposomes. Micelles consist of amphiphillic molecules in a confirmation where the hydrophobic part of the molecule is shielded from the aqueous external phase. Micelles are dependent on a critical concentration to maintain conformation and the types of drugs possible to encapsulate are limited. Gas containing liposomes may in principles carry any payload and due to the gas content they are echogenic and US sensitive. However, gas containing liposomes are generally too large to take advantage of the EPR effect. Hence, efficient passive accumulation of gas containing liposomes in e.g. tumour tissue is not possible at present. Microbubbles are gas bubbles encapsulated by a protein, lipid or phospholipid layer. The gas provides good sonosensitivity, but large size bars the bubbles from efficient EPR effect and possible payloads are restricted. Liposomes can accommodate high drug loads, both of water-soluble and poorly soluble drugs, and their routine clinical use has proven feasible. Also, liposomes can be made in a variety of sizes including small size to accommodate passive tissue accumulation, however, liposomes have not generally been considered to be suitable for US mediated release. Hence, prior art on US sensitive liposomes is rather limited.

Lin & Thomas (Langmuir 2003, vol. 19, no. 4, pp. 1098-1105) report that 100 nm egg yolk phosphatidylcholine (EYPC) liposomes comprising polyethylene glycol (PEG)-grafted lipids (herein referred to as PEG lipid) show enhanced 20 kHz ultrasound sensitivity compared to liposomes with no PEG lipid. The authors tested varying amounts of two different molecular weight PEG lipids (350 Da and 2000 Da) and found that higher mole percents of both PEG lipids correlated positively with US sensitivity. However, the leakage rate levelled off dramatically when the membrane reached about 8 mol % of DPPE-PEG 2000 or about 24 mol % of DPPE-PEG 350, with the smaller PEG species levelling off at a higher absolute leakage rate level (FIG. 3, ibid).

In a later paper Lin & Thomas (Langmuir 2004, vol. 20, no. 15, pp. 6100-6106) further explore the factors affecting ultrasound sensitivity of liposomes. Here, it is shown that for egg yolk PC liposomes there is an inverse relationship between size and ultrasound sensitivity, the latter indicated as release of drug marker (calcein) (ibid, FIGS. 5A and B).

Surprisingly, this trend is reversed when 8 mol % DPPE-PEG 2000 is added to the membrane: Increasing PEG liposome size correlates with increasing US sensitivity (ibid., FIG. 5B). Thus, at sizes below about 50 nm PEGylated liposomes are less sensitive than egg yolk PC liposomes, while the opposite is the case above about 50 nm. In absolute terms, small non-PEGylated liposomes below about 50 nm appear to be superior to any PEGylated liposome in the size range 30-200 nm.

Pong and co-workers (Ultrasonics 2006, vol 45, Issue 1-4, pp. 133-145) investigate the leakage from liposomes in response to high (1-1.6 MHz) and low (20 kHz) frequency ultrasound. In this disclosure liposomes are made of 1,2-diacyl-sn-glycero-3 phosphocholine (PC) and between 0 and 8 mol % DPPE-PEG 2000. PC is a mixture of unsaturated lipids of inhomogeneous acyl chain length isolated from e.g. egg or soy. Pong et al. find, in accordance with Lin & Thomas (supra), that increasing concentrations of DPPE-PEG 2000 improves US mediated release at 20 kHz ultrasound. Furthermore, no improvement is observed above 5 mol % of DPPE-PEG 2000, that is, no difference in release is observed between 5 and 8 mol % of said PEG (FIG. 4, ibid). The authors further report that at 1 MHz US, the leakage or release from PEGylated PC liposomes are positively correlated with size: larger liposomes are more sensitive to US than smaller liposomes.

U.S. Pat. No. 6,123,923 (Unger & Wu) discloses optoacoustic agents and methods for their use. These agents may comprise PEG and saturated phospholipids. However, these agents comprise gases and are of micrometer size, restricting their field if application.

Huang and MacDonald (2004) describes an ultrasound sensitive liposome comprising both saturated and non-saturated phospholipids, as well as an air bubble. The liposome does not contain PEG and the size of the particle is about 800 nm. The ultrasound sensitivity of non-acoustically liposomes is reported to be negligible.

US 2006/0002994 (Thomas, Lin, and Rapoport) reports that 100 nm liposomes consisting of egg yolk PC and PEG have improved ultrasound sensitivity sensitivity compared to egg yolk liposomes without PEG.

In view of the above disclosures the following conclusions may be drawn regarding US mediated release from liposomes:
Lipid-grafted PEG improves release up to a certain concentration, the specific concentration being determined by the molecular weight of the PEG molecule.
Small molecular weight is better than big molecular weight PEG molecules
US sensitivity improves with increasing size in Egg yolk -PEG liposomes.
US sensitivity decreases with increasing size in Egg yolk PC liposomes.

The major challenge within US mediated release is still to design particles showing high US sensitivity, low toxicity, and good biodistribution/pharmacokinetic characteristics. In a recent study conducted by the current applicant, it was shown for the first time that adjuvant ultrasound treatment significantly increased the antitumoural effect of conventional liposomal doxorubicin (CAELYX® or DOXIL®) on tumour growth (Myhr & Moan in Cancer Letters, 232:206-213, 2006)

The current inventors herein disclose novel US sensitive drug delivery particles with surprising properties. Contrary to the above disclosures, the current inventors find that the combination of PEG and small liposome size synergistically improves US sensitivity. The current invention may be used to efficiently deliver drugs in a defined tissue volume to combat localized disease.

### Definitions

‘PC’ herein means 1,2-diacyl-sn-glycero-3 phosphocholine or, in short, phosphatidylcholine.
DPPE-PEGXXXX means 1,2-dipalmitoyl-sn-glycero-3-phosphoethanolamine-N-[methoxy(polyethylene glycol)-XXXX, wherein XXXX signifies the molecular weight of the polyethylene glycol moiety, e.g. DPPE-PEG2000 or DPPE-PEG5000.
PEG herein means polyethyleneglycol and its derivates
PEG lipid means any PEG conjugated lipid or sterol.
‘US’ herein means ultrasound.
‘US sensitive’, ‘sonosensitive’ or ‘acoustically sensitive’ herein means the ability of a particle to release its payload in response to ultrasound.
‘CAELYX®-like liposome’ herein means a liposome with identical membrane composition to the liposome sold under the tradename CAELYX®, except that doxorubicin is exchanged with calcein. Caleyx® consists of 57 mol % HSPC (hydrogenated soy phosphatidyl choline), 38 mol % cholesterol, 5 mol % DSPE-PEG 2000, as well as doxorubicin (present as the hydrochloride). The liposome size (intensity weighted) is measured to between 75 and 80 nm in isosmotic sucrose/HEPES solution (pH 7.4) by the present inventors (Nanosizer, Malvern Instruments, Malvern UK).

All ranges mentioned herein includes the endpoints, that is, the range ‘from 14 to 18’ includes 14 and 18.

The PEG concentrations mentioned herein are nominal values unless otherwise mentioned. Nominal concentration means the concentrations of PEG in the liposome hydration liquid.

### DETAILED DESCRIPTION OF THE INVENTION

The current invention comprises a method of treating a disease or condition comprising the steps of
administering to a patient in need thereof a particulate material of size less than 100 nm comprising a lipid, polyethylene glycol (PEG), and a drug and;
exposing the patient to acoustic energy.

By exposing the patient, preferably only the diseased area, to acoustic energy the drug will escape the particulate material, thus obtaining an increased local concentration of said drug.

The present inventors have found that the combination of small particle size and high PEG content in drug delivery systems comprising phospholipids acts synergistically to produce dramatically improved drug release in response to acoustic energy, e.g. ultrasound. The particulate material may be of any conformation, like a matrix or a membrane, although said material is preferably a membrane. In a preferred embodiment the membrane constitutes a bilayer liposome. Preparation of liposomes are well known within the art and a number of methods may be used to prepare the current material.

The data herein enclosed shows that particle size and PEG concentration acts synergistically at sizes below 100 nm. Accordingly, the size of the particulate material used in the invention should be less than 100 nm, preferably less than 90 nm, more preferably less than 85 nm, more preferably 75 nm or less, or even more preferably 70 nm or less. In a particularly preferred embodiment the size falls within the range 60 to 86 nm, more preferably 60 to 81 nm, more preferably 60 to 74 nm. In a most preferred embodiment the size falls within the range 60 to 64 nm. The current inventors have employed photon correlation spectroscopy to determine size.

The particulate material may comprise any type of lipid, although an amphiphillic lipid is preferred. The lipid or amphiphillic lipid may be e.g. glycerol based (e.g. phospholipids), or a sphingolipid (e.g. ceramides), however, phospholipids are preferred. Furthermore, the lipid is preferably saturated. The particulate material may, however, comprise minor amounts of unsaturated lipid material. In a preferred embodiment of the current invention all lipids are phospholipids, wherein said phospholipids are mainly saturated phospholipids. Particularly, 20 mol % or less of all phospholipids are unsaturated phospholipids, more preferably 10 mol % or less, and even more preferably less than 2 mol %. In a preferred embodiment of the present invention essentially all or all phospholipids of the material are saturated. Hence, the material typically comprises no unsaturated phospholipids, alone or conjugated to other molecules, e.g. PEG. The saturated phospholipid may be of any type and of any source. Nevertheless, the selected lo phospholipids will typically have an acyl chain length within the range of 12 to 20 carbon atoms, preferably within 14 to 18 carbon atoms, more preferably within the range of 16 to 18 carbon atoms. Furthermore, the polar head of the phospholipid may be of any type, e.g. DxPE, DxPC, DxPA, DxPS or DxPG. Neutral phospholipid components of the lipid bilayer are preferably a phosphatidylcholine, most preferably chosen from diarachidoylphosphatidylcholine (DAPC), hydrogenated egg phosphatidylcholine (HEPC), hydrogenated soya phosphatidylcholine (HSPC), distearoylphosphatidylcholine (DSPC), dipalmitoylphosphatidylcholine (DPPC) and dimyristoylphosphatidylcholine (DMPC). Negatively charged phospholipid components of the lipid bilayer may be a phosphatidylglycerol, phosphatidylserine, phosphatidylinositol, phosphatidic acid or phosphatidylethanolamine compound, preferably a phosphatidylglycerol like DPPG. In preferred embodiments of the current invention the saturated non-charged phospholipids are DMPC, DPPC, or DSPC, or any combination thereof. In a most preferred embodiment said non-charged saturated phospholipid is DPPC and/or DSPC, even more preferably DSPC. It is particularly preferred that the acyl chains of all phospholipids comprised in the particulate material are of identical length.

As stated above, PEG is essential to obtain the observed synergistic US mediated drug release effect. PEG may be present in any suitable concentration. However, the particle for use in the current invention preferably comprises at least 1 mol % PEG, more preferably at least 4.5 mol % PEG, even more preferably at least 5.5 mol % PEG, even more preferably at least 8 mol % PEG, even more preferably at least 10 mol % PEG, and most preferably 11.5 mol % or more. Preferably, the PEG content is within the range 1 to 15 mol %, more preferably within the range 4.5 to 11.5 mol %, even more preferably within the range 5.5 to 8 mol %. The PEG molecule may be of any molecular weight or type, however, it is preferred that the molecular weight is 350 Da or more, more preferably 2000 Da or more, even more preferably within the range 2000 to 5000 Da. In a preferred embodiment the molecular weight is 2000 and 5000 Da, more preferably 2000 or 5000 Da. The inventors have showed that sonosensitivity is positively correlated to the molecular weight of the PEG moiety. Hence PEG5000 further improves sonosensitivity compared to PEG2000. The PEG molecule may be associated with any molecule allowing it to form part of the particulate material, like an amphiphilic lipid or sterol. Preferably the PEG molecule is conjugated to a ceramide, phospholipid or sterol. The phospholipid may be DxPE (e.g. DMPE, DPPE, or DSPE), while the sterol may be cholesterol or vitamin D, or any of its derivates. The acyl chain length of the phospholipid should be the same as that of the main saturated phospholipid (PC), as described above. In a preferred embodiment PEG or lipid-grafted PEG is DPPE-PEG 2000, DPPE-PEG 5000, DSPE-PEG 2000 an/or DSPE-PEG 5000. In a particularly preferred embodiment PEG or lipid-grafted PEG is DSPE-PEG 2000 or DSPE-PEG 5000.

The drug may be any drug suitable for the purpose. However, anti-bacterial drugs, anti-inflammatory drugs, anti cancer drugs, or any combination thereof are preferred. As the current technology is particularly adapted for treating cancer, anti cancer drugs are preferred. Anti cancer drugs includes any chemotherapeutic, cytostatic or radiotherapeutic drug.

The general groups of cytostatics are alkylating agents (L01A), anti-metabolites (L01B), plant alkaloids and terpenoids (L01C), vinca alkaloids (L01CA), podophyllotoxin (L01CB), taxanes (L01CD), topoisomerase inhibitors (L01CB and L01XX), antitumour antibiotics (L01D), hormonal therapy. Examples of cytostatics are daunorubicin, cisplatin, docetaxel, 5-fluorouracil, vincristine, methotrexate, cyclophosphamide and doxorubicin.

Accordingly, the drug may include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, and angiogenesis inhibitors.

The drug may also be alpha emitters like radium-223 (223Ra) and/or thorium-227 (227Th) or beta emitters. Other alpha emitting isotopes currently used in preclinical and clinical research include astatine-211 (211At), bismuth-213 (213Bi) and actinium-225 (225Ac).

Moreover, the drug may further comprise anti-cancer peptides, like telomerase or fragments of telomerase, like hTERT; or proteins, like monoclonal or polyclonal antibodies, scFv, tetrabodies, Vaccibodies, Troybodies, etc.

More specifically, therapeutic agents that may be included in the particulate material include abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, zoledronate, and ELACYT™.

The drug is preferably cyclophosphamide, methotrexate, fluorouracil (5-FU); anthracyclines, like e.g. doxorubicin, epirubicin, or mitoxantrone; cisplatin, etoposide, vinblastine, mitomycin, vindesine, gemcitabine, paclitaxel, docetaxel, carboplatin, ifosfamide, estramustine, or any combination thereof; even more preferably doxorubicin, methotrexate, 5-FU, cisplatin, or any combination thereof.

In a preferred embodiment of the current invention the drug is a water soluble drug. In a even more preferred embodiment the drug is doxorubicin.

The particulate material may also comprise a sterol, wherein the sterol may be cholesterol, a secosterol, or a combination thereof. The secosterol is preferably vitamin D or a derivate thereof, more particularly calcidiol or a calcidiol derivate. Preferably, the particulate material comprises 5 to 40 mol % cholesterol, more particularly 10 to 30 mol %, and even more particularly 15 to 25 mol % cholesterol. In preferred embodiments of the current invention the particulate material comprises 20, 25 or 40 mol % cholesterol.

Furthermore, the particulate material may comprise magnetic resonance imaging (MRI) contrast agents as described in international applications WO 2008/033031 and WO 2008/035985, fully incorporated herein by reference.

The localized disease may be any disease in need of local treatment. Infective, viral, inflammatory and neoplastic diseases are preferred. Infective disease may be of bacterial, viral, parasitic, or fungal origin. Localized cancers are of particular interest, principally, cancers of head and neck, skin, breast, liver, prostate, as well as sarcomas. It should be noted that the current particles naturally accumulate in liver, skin, spleen, tumours and inflammations and are therefore especially well-suited to treat the above diseases. In addition, acoustic energy (e.g. ultrasound) is easily deposited in the mentioned tissues.

The drug payload of the US sensitive material is released by means of acoustic energy, e.g. ultrasound. In this way the patient is protected against potential toxic effects of the drug en route to the target tissue, where high local concentrations of the drug are obtainable. The ultrasound frequency is preferably below 3 MHz, more preferably below 1.5 MHz, even more preferably below 1 MHz, within the range 20 kHz to 1 MHz, within the range 20 kHz to 500 kHz, within the range 20 kHz to 100 kHz. In a preferred embodiment of the current invention the frequency is 20 kHz. It should, however, be noted that focused ultrasound transducers may be driven at significantly higher frequencies than nonfocused transducers and still induce efficient drug release from the current sonosensitive material. Without being limited to prevailing scientific theories, the current inventors believe that the level of ultrasound induced cavitation in the target tissue is the primary physical factor inducing drug release from the particulate material of the invention. A person skilled in the art of acoustics would know that ultrasound at any frequency may induce so-called transient or inertial cavitation. Hence, the specific frequency is not essential for the current invention as long as the acoustic energy sufficient to induce drug release.

The current invention also comprises an ultrasound sensitive particulate material as used and described in the method supra.

The particulate material as described anywhere supra does not comprise so-called microbubbles, that is, lipid coated air bubbles of e.g. perfluorobutane or perfluoropropane is gas. As mentioned above these entities are too large to take advantage of the EPR effect, a general predicament of all air or gas filled drug delivery particles. Furthermore, it has so far been assumed that gas was necessary to make drug carriers acoustically sensitive. It is a main point of the current disclosure that liposomes can be made acoustically sensitive in the absence of gas. Typically, the particulate material as described anywhere supra will not comprise air bubbles of perfluorobutane or perfluoropropane gas, or any non-dissolved gases. Hence, in a preferred embodiment of the current invention said particulate material comprises no non-dissolved gases.

The current invention further comprises a composition comprising the above US sensitive particulate material.

The current invention also comprises a pharmaceutical composition comprising the above US sensitive particulate material.

Another aspect of the current invention is a method of manufacturing the particulate material described supra comprising the steps of producing inhomogeneous population of particulate material comprising a drug, chemical, or buffer of interest, further process said population to form particles of size below 100 nm with one phospholipid bilayer. Also, the current invention comprises a kit comprising the particulate material of the invention.

### DESCRIPTION OF THE DRAWINGS

FIG. 1. CAELYX® liposomes exposed to 20 kHz ultrasound over a period of 6 minutes. Percent doxorubicin release is measured after 0, 1, 2, 4, and 6 minutes of ultrasound exposure.

FIG. 2. CAELYX®-like liposomes exposed to 20 kHz ultrasound over a period of 6 minutes. Percent calcein release is measured after 0, 1, 2, 4, and 6 minutes of ultrasound exposure.

FIG. 3. A selection of five liposomal formulations of calcein from a multivariate study (CCD1) exposed to 20 kHz ultrasound over a period of 6 minutes. The release profile is compared to CAELYX®-like liposomes. Percent calcein release is measured after 0, 1, 2, 4, and 6 minutes of ultrasound exposure.

FIG. 4. A selection of two liposomal formulations of calcein from a multivariate study (CCD1) exposed to 20 kHz ultrasound over a period of 6 minutes. The release profile is compared to CAELYX®-like liposomes. Percent calcein release is measured after 0, 1, 2, 4, and 6 minutes of ultrasound exposure.

FIG. 5. Regression coefficients of CCD1 data at 1 minute US exposure. From left to right: Size, DPPG, DPPE-PEG 2000, cholesterol, acyl chain length of main saturated PC (DMPC, DPPC, or DSPC), size*DPPE-PEG 2000.

FIG. 6. Surface plot of percent US mediated release as a function of liposome size (nm) and DPPE-PEG 2000 content (mol %). A clear synergy is observed between size and PEG content.

FIG. 7. Regression model (US1 min), SEC=9,3.

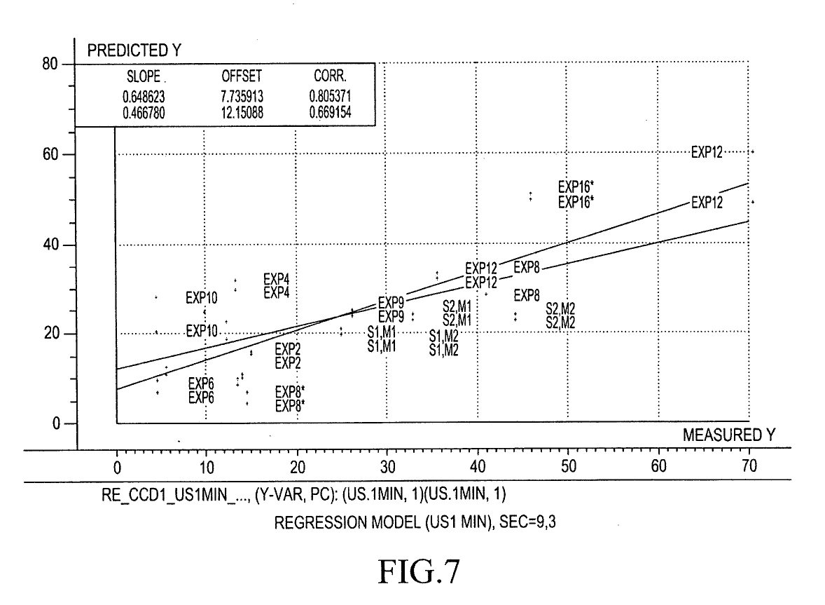

### EXAMPLES

### Example 1

### Preparation of Liposomes

DMPC, DPPC, DSPC, DPPG and DPPE-PEG 2000 were purchased from Genzyme Pharmaceuticals (Liestal, Switzerland). Cholesterol was obtained from Sigma Aldrich.

Calcein liposomes were prepared according to the thin film hydration method (D. D. Lasic “Preparation of liposomes”, in Lasic D D editor, Liposomes from Physics to Applications. Amsterdam Elsevier Science Publishers BV, the Netherlands, 1993, p. 67-73). Liposomes were loaded with calcein via passive loading, the method being well known within the art.

Extraliposomal calcein was removed by exhaustive dialysis. Liposome dispersion contained in disposable dialysers (MW cut off 100 000 D) and protected from light was dialysed at room temperature against an isosmotic sucrose solution containing 10 mM HEPES and 0.02% (w/v) sodium azide solution (representing extraliposomal phase) until acceptable residual level of calcein resulted. The liposome dispersion was then, until further use, stored in the fridge protected from light.

### Example 2

### Characterisation of Liposomes

Liposomes were characterised with respect to key physicochemical properties like particle size, pH and osmolality by use of well-established analytical methodology.

The mean particle size (intensity weighted) and size distribution were determined by photon correlation spectroscopy at a scattering angle of 173° and 25 deg C. (Nanosizer, Malvern Instruments, Malvern, UK). The width of the size distribution is defined by the polydispersity index. Prior to sample measurements, a latex standard (60 nm) was run. Sample preparation consisted of 10 μL of liposome dispersion being diluted with 2 mL particle free isosmotic sucrose solution containing 10 mM HEPES (pH 7.4) and 0.02% (w/v) sodium azide. Sample triplicates were analysed.

Osmolality was determined on non-diluted liposome dispersions by freezing point depression analysis (Fiske 210 Osmometer, Advanced Instruments, MS., US). Prior to sample measurements, a reference sample with an osmolality of 290 mosmol/kg was measured; if not within specifications, a three step calibration was performed. Duplicates of liposome samples were analysed.

### Example 3

### US Mediated Release Methodology

Liposomes were exposed to 20 kHz ultrasound up to 6 min. in a custom built sample chamber as disclosed in Huang and MacDonald (Biochimica et Biophysica Acta, 2004, 1665: 134-141). The US power supply and converter system was a ‘Vibra-Cell’ ultrasonic processor, VC 750, 20 kHz unit with a 6.35 cm diameter transducer, purchased from Sonics and Materials, Inc. (USA). Pressure measurements were conducted with a Bruel and Kjaer hydrophone type 8103.

The system was run at the lowest possible amplitude at 20% of maximum amplitude. This translates to a transducer input power of 0.9-1.2 W/cm². At this minimal amplitude pressure measurements in the sample chamber gave 85-95 kPa.

The release assessment of calcein or doxorubicin is based on the following well-established methodology: Intact liposomes containing calcein or doxorubicin will display low fluorescence intensity due to self-quenching caused by the high intraliposomal concentration of material. Ultrasosund mediated release of material into the extraliposomal phase can be determined by a marked increase in fluorescence intensity due to a reduced quenching effect. The following equation is used for release quantification:

$$\% release = \frac{( {F}_{u} - {F}_{b} )}{( {F}_{T} - {F}_{b} )} \times 100$$

Where F_{b} and Fᵤ are, respectively, the fluorescence intensities of the liposome sample before and after ultrasound application. F_{T} is the fluorescence intensity of the liposome sample after solubilisation with surfactant. Studies have shown that the solubilisation step must be performed at high temperature, above the phase transition temperature of the phospholipid mixture.

Fluorescence measurements were undertaken with a Luminescence spectrometer model LS50B (Perkin Elmer, Norwalk, Conn.) equipped with a photomultiplier tube R3896 (Hamamatsu, Japan). Fluorescence measurements are well known to a person skilled in the art.

### Example 4

### CAELYX® in vitro US Sensitivity

Liposomal doxorubicin is marketed under the tradename DOXIL® in the American market and CAELYX® in the European market. The tradename CAELYX® shall be used in the current document.

CAELYX® was obtained from the pharmacy at the Norwegian Radium Hospital (Oslo, Norway). CAELYX® consists of 57 mol % HSPC (hydrogenated soy phosphatidyl choline), 37 mol % cholesterol, 5 mol % DSPE-PEG 2000, as well as doxorubicin. The liposome size (intensity weighted) is measured to between 75 and 80 nm in isosmotic sucrose/HEPES solution (pH 7.4) by the present inventors (Nanosizer, Malvern Instruments, Malvern UK). However, others, including inventor and producer Alza/Johnson & Johnson of DOXIL®), report a size of 100 nm (FDA prescribing information, Gabizon et al Cancer Research 1994, Drummond et al Pharmacological Reviews 1999).

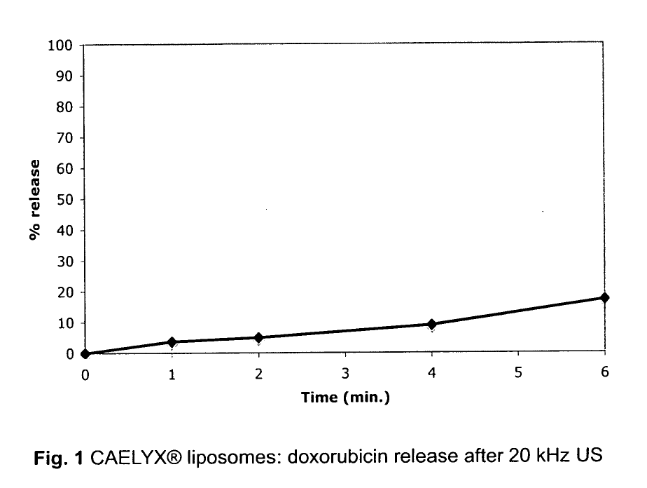

CAELYX® diluted 1:100 in isosmotic and isoprotic sucrose/HEPES solution was exposed to 20 kHz in the US chamber and release was estimated at 0, 1, 2, 4, and 6 minutes according to the method above (FIG. 1). The US settings were as described above. The data showed 3.7% release at 1 min, 5% at 2 min, and 17.2% at 6 minutes.

### Example 5

### US Sensitivity of CAELYX®-Like Liposomes

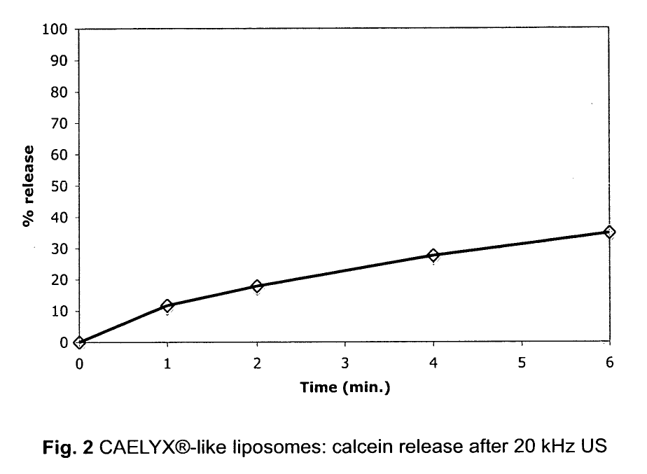

A liposome with membrane constituents identical with CAELYX®, but loaded with the fluorescent marker calcein was exposed to US as described in Example 4. The data show that CAELYX®-like liposomes carrying calcein are more sensitive to US than CAELYX® (FIG. 2). At 2 minutes the release from the calcein containing CAELYX®-like liposomes is 17.9% compared to 5% for the CAELYX® liposome of Example 4. This may be due to the fact that doxorubicin is in a precipitated crystalline state within the liposome, while calcein is in dissolved state.

### Example 6

### Liposome Formulations (CCDI Study)

A number of liposomal formulations of calcein were manufactured to investigate the impact of varying amounts of cholesterol, DPPE-PEG, DPPG, as well as different acyl chain lengths of the main saturated phospholipid (PC) on liposome sonosensitivity. The formulations were designed to take advantage of biometry and multivariate data analysis. The chemical constitution of the formulations are summarised in Table 1 in mol %. All values are nominal values, that is, the amount used in thin film production.

TABLE 1
Formulation Overview CCDI study

| | Size | | | | | DPPE- | DPPE- | |
|---|---|---|---|---|---|---|---|---|
| Formulation | (nm) | DPPG | DMPC | DPPC | DSPC | PEG2000 | PEG5000 | Cholesterol |
| 1 | 117 | 0 | 99 | | | 1 | | 0 |
| 2 | 70.9 | 0 | 59 | | | 1 | | 40 |
| 2* | 98 | 0 | 69 | | | 1 | | 40 |
| 3 | N/A | 0 | 90 | | | 10 | | 0 |
| 4 | 88.2 | 0 | 50 | | | 10 | | 40 |
| 5 | 72.8 | 0 | | | 99 | 1 | | 0 |
| 6 | 106 | 0 | | | 59 | 1 | | 40 |
| 7 | 85.1 | 0 | | | 90 | 10 | | 0 |
| 8 | 73.9 | 0 | | | 50 | 10 | | 40 |
| 8* | 110 | 0 | | | 50 | 10 | | 40 |
| 9 | 62.8 | 10 | 89 | | | 1 | | 0 |
| 10 | 107 | 10 | 49 | | | 1 | | 40 |
| 11 | N/A | 10 | 80 | | | 10 | | 0 |
| 12 | 60.1 | 10 | 40 | | | 10 | | 40 |
| 12* | 92 | 10 | 40 | | | 10 | | 40 |
| 13 | 94.7 | 10 | | | 89 | 1 | | 0 |
| 14 | 70.5 | 10 | | | 49 | 1 | | 40 |
| 15 | 94.8 | 10 | | | 80 | 10 | | 0 |
| 16 | 91.4 | 10 | | | 40 | 10 | | 40 |
| 16* | 64 | 10 | | | 40 | 10 | | 40 |
| S1 | 90 | 5 | | 69.5 | | 5.5 | | 20 |
| S2 | 81 | 5 | | 69.5 | | 5.5 | | 20 |
| S3 | 88 | 5 | | | 69.5 | 5.5 | | 20 |
| S4 | 101 | 5 | | | 69.5 | 5.5 | | 20 |

### Example 7

### US Release Study of CCD1 Liposomes

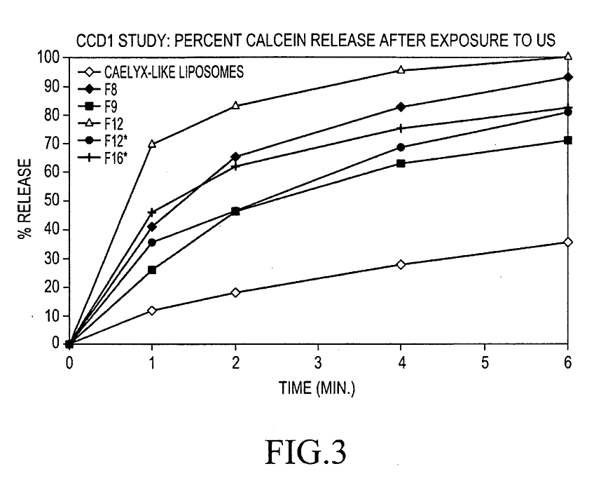

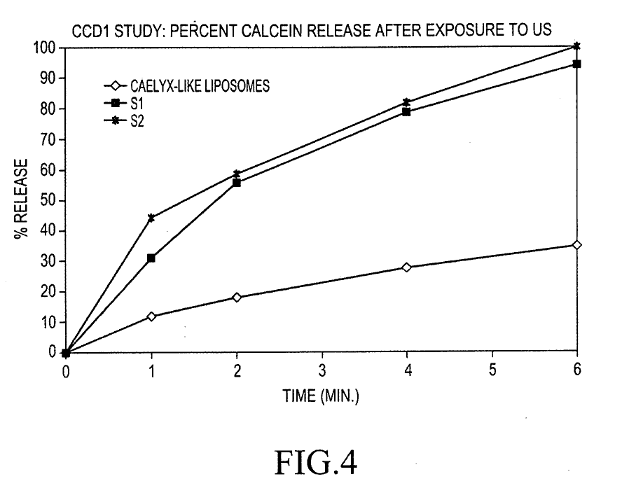

The sonosensitivity and release properties of the CCD1 liposomes were tested in the in vitro experimental set-up as described above. All experiments were conducted at least twice. The results of the experiments are summarized in Table 2. At 2 minutes US exposure formulations 8, 9, 12, 12*, 16* (FIG. 3), S1, S2 (FIG. 4) show particular sensitivity compared to the CAELYX®-like liposome. It should be noted that all phospholipids of S1 and S2 have identical acyl chain length.

TABLE 2
Percent release after 20 kHz US exposure (CCD1 study)

| Formulation | US 1 min | US 2 min | US 4 min | US 6 min |
|---|---|---|---|---|
| 1 | 61.1 | 85 | 100 | 100 |
| 2 | 9.9 | 15.9 | 25.9 | 32.9 |
| 2* | 15 | 21.5 | 34.1 | 44.4 |
| 3 | N/A | N/A | N/A | N/A |
| 4 | 13.2 | 27.4 | 48.6 | 65.9 |
| 5 | 13.5 | 18.4 | 29.1 | 36.6 |
| 6 | 4.6 | 8.8 | 13.2 | 16.7 |
| 7 | 15.9 | 29.1 | 46.2 | 63.5 |
| 8 | 40.9 | 64.9 | 82.3 | 92.4 |
| 8* | 14.5 | 22.8 | 37 | 47.8 |
| 9 | 26.1 | 46.0 | 62.6 | 70.4 |
| 10 | 4.5 | 10.1 | 18.7 | 26.7 |
| 11 | N/A | N/A | NA/ | N/A |
| 12 | 70.3 | 83.3 | 95.3 | 100.0 |
| 12* | 35.5 | 46.3 | 68.1 | 80.3 |
| 13 | 5.6 | 9.8 | 19.4 | 26.7 |
| 14 | 12.5 | 18.6 | 25.2 | 30.0 |
| 15 | 12.2 | 24.1 | 39.3 | 54.0 |
| 16 | 10.6 | 19.2 | 35.3 | 48.8 |
| 16* | 45.8 | 61.6 | 74.7 | 81.9 |
| S1 | 31.2 | 55.9 | 78.8 | 93.8 |
| S2 | 44.1 | 58.3 | 81.6 | 100 |
| S3 | 19.8 | 36.3 | 57.8 | 76.4 |
| S4 | 17.2 | 36.3 | 62.4 | 81.4 |

### Example 8

### Multivariate Analysis and Biometry

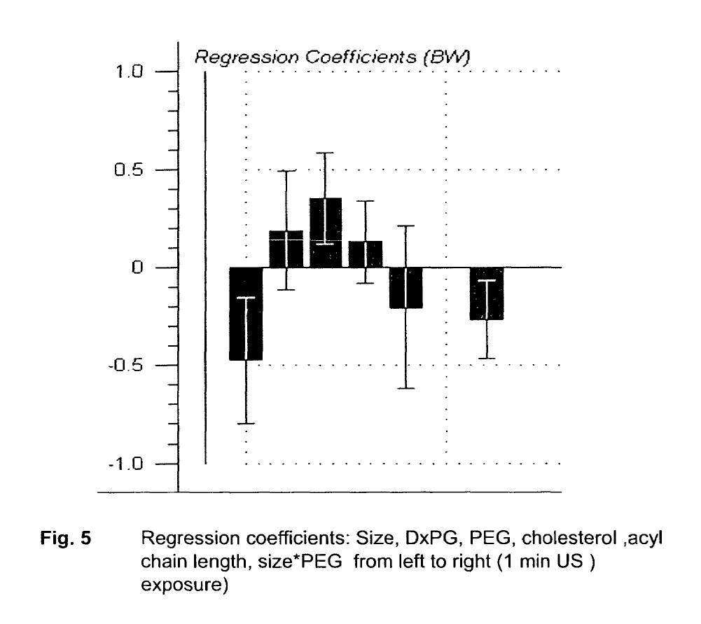

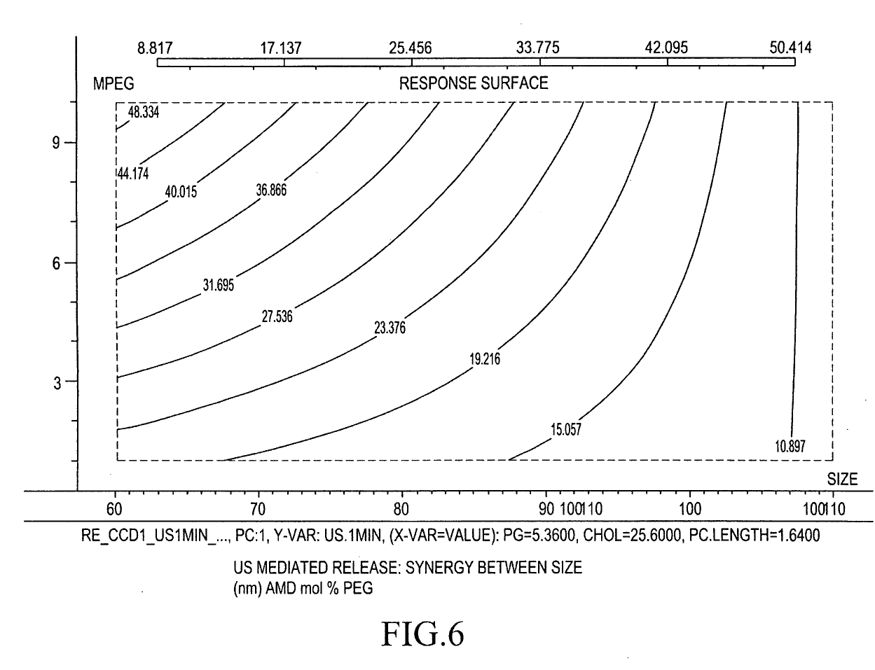

Multivariate analysis of the data of Example 7 showed that there was a positive correlation between mol % lipid-grafted PEG and sonosensitivity and a negative correlation between liposome size and sonosensitivity (FIG. 5), that is, smaller liposomes are more sonosensitive. Moreover, the analysis showed synergy between lipid-grafted PEG and size: Small liposomes with high levels of PEG had unprecedented and unexpected high sonosensitivity (FIG. 6). All correlations have statistical significance. It was also observed a positive trend correlation between DPPG and cholesterol content, respectively (FIG. 5).

### Example 9

### Liposome Formulations (CCD2 Study)

In a second study design cholesterol and lipid-grafted PEG content was varied in liposomes with a target size of 85±10 nm in size to further investigate liposome sonosensitivity. The chemical constitution of the formulations are summarised in Table 3 in mol %. All values are nominal values, that is, the amount used in thin film production.

TABLE 3
Formulation overview CCD2 study

| Formu- | | | DPPE- | DPPE- | DSPE- | Choles- |
|---|---|---|---|---|---|---|
| lation | Size | DSPC | PEG5000 | PEG2000 | PEG2000 | terol |
| CCD2 1 | 97 | 78 | 4.5 | | | 17.5 |
| CCD2 2 | 87 | 71 | 11.5 | | | 17.5 |
| CCD2 3 | 98 | 63 | 4.5 | | | 32.5 |
| CCD2 4 | 89 | 56 | 11.5 | | | 32.5 |
| CCD2 5 | 86 | 67 | 8 | | | 25 |
| CCD2 6 | 96 | 67 | 8 | | | 25 |
| CCD2 7 | 87 | 60 | 15 | | | 25 |
| CCD2 7* | 142 | 60 | 15 | | | 25 |
| CCD2 8 | 102 | 52 | 8 | | | 40 |
| CCD2 8* | 88 | 52 | 8 | | | 40 |
| CCD2 9 | 85 | 82 | 8 | | | 10 |
| CCD2 9* | 128 | 82 | 8 | | | 10 |
| CCD2 10 | 89 | 74 | 1 | | | 25 |
| CCD2 11 | 83 | 67 | | 8 | | 25 |
| CCD2 12 | 85 | 60 | | 15 | | 25 |
| CCD2 13 | 86 | 52 | | 8 | | 40 |
| CCD2 14 | 83 | 82 | | 8 | | 10 |
| CCD2 15 | 81 | 74 | | 1 | | 25 |
| CCD2 16 | 88 | 67 | | | 8 | 25 |
| CCD2 17 | 84 | 60 | | | 15 | 25 |
| CCD2 18 | | 52 | | | 8 | 40 |
| CCD2 19 | | 82 | | | 8 | 10 |
| CCD2 20 | | 74 | | | 1 | 25 |

### Example 10

### US Release Study of CCD2 Liposomes

The sonosensitivity and release properties of the CCD2 liposomes were tested in the in vitro experimental set-up as described above. All experiments were conducted twice. The results of the experiments are summarized in Table 4.

TABLE 4
Percent release after 20 kHz US exposure (CCD2 study)

| | US 0.5 | | | | |
|---|---|---|---|---|---|
| Formulation | min | US 1 min | US 1.5 min | US 2 min | US 6 min |
| CCD2 1 | 6.9 | 13.5 | 19.7 | 25.6 | 67.2 |
| CCD2 2 | 9.5 | 19.1 | 27.3 | 35.2 | 81.2 |
| CCD2 3 | 10.3 | 17.6 | 24.5 | 31.2 | 67.6 |
| CCD2 4 | 8.7 | 17.5 | 25.6 | 32.8 | 67.9 |
| CCD2 5 | 10.9 | 21.7 | 30.3 | 38.3 | 81.3 |
| CCD2 6 | 9.9 | 20.2 | 27.7 | 36.7 | 73.1 |
| CCD2 7 | 17.1 | 28.4 | 38.4 | 46.7 | 91.4 |
| CCD2 7* | 12.7 | 24.1 | 33.0 | 40.7 | 82.8 |
| CCD2 8 | 9.2 | 19.1 | 28.0 | 35.2 | 79.5 |
| CCD2 8* | 13.5 | 25.6 | 32.8 | 39.6 | 77.7 |
| CCD2 9 | 6.7 | 17.2 | 25.8 | 33.1 | 76.7 |
| CCD2 9* | 9.4 | 15.7 | 25.8 | 41.8 | 82.9 |
| CCD2 10 | 4.4 | 10.0 | 14.9 | 20.5 | 46.8 |
| CCD2 11 | 10.6 | 18.0 | 24.8 | 30.6 | 57.2 |
| CCD2 12 | 22.4 | 34.2 | 41.7 | 48.1 | 83.4 |
| CCD2 13 | 9.9 | 16.1 | 20.3 | 24.3 | 58.8 |
| CCD2 14 | 8.6 | 17.2 | 24.7 | 32.3 | 64.3 |
| CCD2 15 | 7.7 | 13.3 | 18.2 | 22.8 | 46.3 |
| CCD2 16 | 8 | 15 | 20 | 25 | 49 |
| CCD2 17 | 10 | 19 | 29 | 35 | 68 |

### REFERENCES

Huang & MacDonald, Acoustically active liposomes for drug encapsulation and ultrasound-triggered release. Biochimica et Biophysica Acta, 1664 (2004) pages 134-141.
Kheirolomoom et al, Acoustically-active microbubbles conjugated to liposomes: Charaterization of a proposed drug deivery vehichle. Journal of Controlled Release, 2007.
Kono K. Takagishi. Temperature-sensitive liposomes. Methods in Enzymology, 387:73-82, 2004
Larina I V. Evers B M. et al. Enhancement of drug delivery in tumors by using interaction of nanoparticles with ultrasound radiation, Technol. Cancer Res. Treat. 4:217-226, 2005
Lin & Thomas, Langmuir, 2004, vol. 20, no. 15, pp. 6100-6106.
Lin & Thomas, Langmuir, 2003, vol. 19, no. 4, pp. 1098-1105.
Lokling K E. Fossheim S L. et al. Biodistribution of pH-responsive liposomes for MRI and a novel approach to improve the pH-responsiveness, J. Control. Release. 98:87-95, 2004
Maeda H. Matsumura Y. Tumoritropic and lymphotropic principles of macromolecular drugs. Crit. Rev. Ther. Drug Carrrier Syst, 6:193-210, 1989.
Myhr G. Moan J. Synergistic and tumour selective effects of chemotherapy and ultrasound treatment, Cancer Letters, 232:206-213, 2006
Pitt et al. Ultrasonic Drug Delivery—A General Review, Expert Opin. Drug Deliv. 2004, 1 (1), pp. 37-56.
Pong et al. Ultrasonics 2006, vol 45, Issue 1-4, pp. 133-145.
Slepushkin V. Simoes S. et al. Sterically stabilised pH sensitive liposomes. Methods in Enzymology, 387:134-146, 2004.
U.S. Patent Application Publication No. 2006/02994A1
U.S. Pat. No. 6,033,646 A
U.S. Pat. No. 6,123,923

We claim:

1. A method of treating a disease or a condition in a patient in need thereof comprising the steps of
administering to said patient a particulate material of size less than 100 nm comprising a lipid, polyethylene glycol (PEG), and a drug, wherein said lipid is saturated, and;
exposing said patient to acoustic energy.

2. The method of claim 1, wherein the size of the particulate material is 90 nm or less.

3. The method of claim 1, wherein the saturated lipid has an acyl chain length within the range of 14 to 18 carbon atoms.

4. The method of claim 1, wherein the saturated lipid has an acyl chain length within the range of 16 to 18 carbon atoms.

5. The method of claim 1, wherein all lipid acyl chains present in the particulate material are of identical length.

6. The method of claim 1, wherein the saturated lipid is dipalmitoylphosphatidylcholine (DPPC) and/or distearoylphosphatidylcholine (DSPC).

7. The method of claim 1, wherein the PEG content is higher than 5.5 mol % based on the total mol % of the particulate material.

8. The method of claim 1, wherein the PEG has a molecular weight of 2000 Da or higher.

9. The method of claim 1, wherein said material does not comprise non-dissolved gas.

10. The method of claim 1, wherein the disease or condition is an infection, an inflammation or a cancer.

11. The method of claim 1, wherein the acoustic energy has a frequency below 1.5 Mhz.

12. An ultrasound sensitive particulate material comprising a lipid, 5.5 mol % or more polyethylene glycol (PEG) and a drug, wherein said material has a size less than 100 nm, and wherein said lipid is saturated.

13. The method of claim 12, wherein acyl chains of said phospholipid have a length within the range of 14 to 18 carbon atoms.

14. The material of claim 12, wherein acyl chains of said phospholipid have a length within the range of 16 to 18 carbon atoms.

15. The material of anyone of claim 12, wherein all phospholipid acyl chains present in the material are of identical length.

16. The material of claim 12, wherein the phospholipid is dipalmitoylphosphatidylcholine (DPPC) and/or distearoylphosphatidylcholine (DSPC).

17. The material of claim 12, wherein the PEG has a molecular weight of 2000 Da or higher.

18. The material of claim 12, wherein said material does not comprise non-dissolved gas.
